(19) United States
(12) Patent Application Publication
(10) Pub. No.: US 2008/0275121 A1
(43) Pub. Date: Nov. 6, 2008

(54) N-HALOGENATED AMINO ACID FORMULATIONS AND METHODS FOR CLEANING AND DISINFECTION

(75) Inventors: Masood A. Chowhan, Arlington, TX (US); Wesley Wehsin Han, Arlington, TX (US); L. Wayne Schneider, Crowley, TX (US); David W. Stroman, Irving, TX (US); Nissanke L. Dassanayake, Fort Worth, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/112,331

(22) Filed: Apr. 30, 2008

Related U.S. Application Data
(60) Provisional application No. 60/970,634, filed on Sep. 7, 2007.
(60) Provisional application No. 60/915,291, filed on May 1, 2007.

(51) Int. Cl. A61K 31/195; A61P 43/00

(52) U.S. Cl. 514/561

(57) ABSTRACT

The present invention relates to methods for disinfecting or cleaning a contact lens comprising contacting a contact lens with a formulation comprising a N-halogenated amino acid and a phase transfer agent for a time sufficient to disinfect or clean the lens. This specification further discloses a formulation for disinfecting a contact lens comprising an N-halogenated amino acid and a phase transfer agent.

### CROSS-REFERENCE TO RELATED APPLICATION

This application claims priority under 35 U.S.C. §119 to U.S. Provisional Patent Application No. 60/970,634 filed Sep. 7, 2007, and to U.S. Provisional Patent Application No. 60/915,291 filed May 1, 2007, both of which are incorporated herein by reference in their entirety.

### TECHNICAL FIELD OF THE INVENTION

The present invention relates to methods for cleaning and disinfecting contact lenses using N-halogenated amino acids. The present invention further relates to formulations for contact lens cleaning and disinfection comprising N-halogenated amino acids and a phase transfer agent.

### BACKGROUND OF THE INVENTION

Ophthalmic contact lenses are exposed to a broad spectrum of microbes and non-infectious contaminants during normal wear. Cleaning and disinfection of lenses is required to avoid the buildup of infectious and non-infectious contaminants on the contact lens surfaces. Daily cleaning and disinfection may be necessary, particularly for hydrophilic (soft) contact lenses. The failure to clean and disinfect lenses properly has consequences for a lens wearer ranging from eye irritation to serious infections. Ocular infections caused by particularly virulent microbes, such as P. aeruginosa, can lead to loss vision if left untreated or if allowed to reach an advanced stage before treatment is initiated.

There is an ongoing need for improved contact lens cleaning and disinfection systems which: 1) are simple to use, 2) have potent antimicrobial activity, and 3) are nontoxic (i.e., do not cause ocular irritation as the result of binding to the lens material). Known techniques for disinfecting and cleaning contact lenses include thermal methods that require time-consuming heating steps. However, because of their convenience, chemical disinfection methods are more widely used in current practice.

In currently known chemical disinfection and cleaning methods, contact lenses are immersed in a liquid formulation for a period of time sufficient to disinfect and clean the lens. To keep the chemical and optical properties of contact lenses unchanged and to assure a low incidence of contact lens user side effects, contact lenses are disinfected using a liquid agent in which a disinfectant is contained in a relatively low concentration. Unfortunately, while the use of formulations having low concentrations of a disinfectant compound generally helps to reduce the potential for undesirable effects, this practice increases the risk that the formulation may not achieve the required level of disinfectant activity. Also, microbial resistance can develop if disinfectant compounds are not used at a sufficient concentration. Therefore, improved formulations for the disinfection of contact lenses are desirable that utilize decreased concentrations of antimicrobial compound components while maintaining sufficient disinfectant activity, reducing the incidence and risk of undesired side effects and microbial resistance.

There is also a need for an improved means of preserving pharmaceutical compositions from microbial contamination. This need is particularly prevalent in the fields of ophthalmic and otic compositions. The antimicrobial utilized to preserve aqueous ophthalmic and otic compositions must be effective in preventing microbial contamination of the compositions when used at concentrations that are non-toxic to ophthalmic and otic tissues.

Some antimicrobial compounds are chlorine-containing, and chlorine, either by itself or in compound form is used for disinfection applications such as the treatment of water supplies. Chlorine compounds with antimicrobial activity include N-chloroamides and imides, chlorocynauric acid and its salts, chloroamine T, 1,3-dichlorohydantoin and N-chloroalkyl amines. Many of these compounds have limited stability, limiting the shelf life of any formulation including them. Other chlorine-containing antimicrobials have been studied as well, such as the chloramines. Weil and Morris studied the reaction between hypochlorite and methylamine and dimethylamine, and discussed the processes by which the chloramines were formed.

### BRIEF SUMMARY OF THE INVENTION

The present invention is directed in certain embodiments to improved methods and formulations for disinfecting and cleaning contact lenses. The improvement is achieved though the use of formulations comprising an N-halogenated amino acid and a phase transfer agent, as described herein. Government regulations require that formulations for disinfecting contact lenses be capable of achieving disinfection without assistance from other compositions (e.g., cleaning compositions or preserved saline rinsing solutions). These regulations have created a need for formulations having significantly greater antimicrobial activity. Many embodiments of the present invention provide contact lens disinfecting formulations having antimicrobial activity sufficient to satisfy this standard. Combining an N-halogenated amino acid and a phase transfer agent increases the biocidal efficacy of the formulations of the present invention and allows a reduction in the concentration of the N-halogenated amino acid. Accordingly, the potential for formulations of the present invention causing ocular irritation is reduced. Also, while not wishing to be bound by theory, it is believed that the formulations of the present invention have increased hydrophobicity thereby increasing their biocidal activity and uptake into contact lenses.

Certain embodiments of the present invention comprise formulations that are free from alpha hydroxyl compounds like citrates which are commonly incorporated in disinfectant solutions for protein removal. Citrate in certain of these formulations may be replaced with such compounds as acetates, adipates, succinates, and/or meleates, especially those with multi carboxylic groups.

One embodiment of the present invention is a method for disinfecting and/or cleaning a contact lens comprising contacting a contact lens with a formulation comprising an N-halogenated amino acid and a phase transfer agent for a time sufficient to disinfect and/or clean the lens.

Another embodiment of the present invention is a formulation for disinfecting a contact lens comprising an N-halogenated amino acid and a phase transfer agent. Yet another embodiment is a pharmaceutical composition comprising an N-halogenated amino acid and a phase transfer agent in a quantity sufficient to preserve the composition.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention.

### DETAILED DESCRIPTION OF THE INVENTION

### I. Definitions

Unless defined otherwise, technical and scientific terms used herein have the same meaning as commonly understood by one of ordinary skill in the art.

As used here, the term “clean” or “cleaning” means to loosen or remove contact lens deposits and surface and subsurface contaminants.

As used herein, the terms “disinfect”, “disinfecting”, and “disinfection” refers to killing or inhibiting the growth of microbes (to include, without limitation, bacteria, viruses, yeast, fungi, spores, protozoa, parasites, etc.).

As used herein, the term “disinfectant” and “antimicrobial” refers to a compound having the ability to kill or inhibit the growth of microbes (to include, without limitation, bacteria, viruses, yeast, fungi, spores, protozoa, parasites, etc.).

As used herein, the term “ion pairing agent” refers to any compound that forms an ion pair with an N-halogenated amino acid in solution.

As used herein, the term “phase transfer agent” refers to any compound that increases the solubility of an N-halogenated amino acid in organic solution. Phase transfer agents include, but are not limited to, ion pairing agents. Phase transfer agents increase the apparent permeability of N-halogenated amino acids when formulated together in solution.

### II. Methods and Formulations

The N-halogenated amino acids of the present invention have the following general formula:

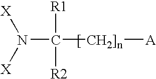

where X is one or more halogens and R1 and R2 are any of the nonpolar, uncharged polar, and charged polar amino acid and amino acid derivative side chains known to those of skill in the art. A represents an acid such as a carboxylic, sulfonic, phosphoric, boric or other acid known to those of skill in the art. There may be one or more carbon atoms between the amine and acid, and each carbon may contain one or more R substituents.

The preferred N-halogenated amino acids of the present invention have the following structure: haloamino-stabilizer-linker-acid, where (a) the “haloamino” is either N-halogen or N,N-dihalogen (e.g., —NHCl or —NCl₂); (b) the “stabilizer” comprises sidechains attached to the carbon next to the haloamino group (e.g., hydrogen, —CH₃, lower alkyl, the group —COOH or a C₃₋₆ cycloalkyl ring); (3) the “linker” is either alkyl or cycloalkyl; and (d) the “acid” is one of the following: —COOH, —SO₃H, —P(═O)(OH)₂, —B(OH)₂ or hydrogen, and all the pharmaceutically acceptable salts of these acids generally known to those skilled in the art, including but not limited to sodium, potassium, calcium, etc.

The most preferred N-halogenated amino acids are 2,2-dimethyl-N,N-dichlorotaurine, analogs of 2,2-dimethyl-N,N-dichlorotaurine formed by replacement of the sulfonic acid group with carboxylic acid, phosphoric acid, borate, etc., 2,2-di alkyl-N,N-dichlorotaurine, and 2,2-R—N,N-dichlorotaurine, where R is an aliphatic or aromatic side chain. Methyl groups of N-halogenated amino acids may be replaced with alkyl, aryl, benzyl, or other hydrocarbon cyclic or non-cyclic groups. Additional N-halogenated amino acids are disclosed in U.S. Provisional Patent Application No. 60/915,291, filed May 1, 2007, entitled “N-HALOGENATED AMINO ACID FORMULATIONS”, the contents of which are incorporated by reference in their entirety.

Generally, the phase transfer agents of the present invention have a basic structure with a head group and lipophilic alkyl chains or aryl substituents. The majority of these phase transfer agents are made from natural building blocks such as fatty acids and alcohols. The total lipophilic alkyl and aryl substituents normally contain a total of about 4-8 carbons to about 30 carbons. The most preferred total number of carbons of the alkyl and aryl substituents is from about 15 to 20 carbons.

The preferred phase transfer agents of the present invention are quaternary amine compounds and include, but are not limited to tetrabutylammonium hydroxide (TBAH), tetrapropylammonium hydroxide (TPAH), hexadecyltrimethylammonium hydroxide, dodecyltriethylammonium hydroxide, tetrabutylphosphonium chloride (TBPC), and combinations thereof. Also included are the various salts of quaternary amine compounds known to those skilled in the art. These include but are not limited to chloride, bromide, sulfate, phosphate, and acetate.

Other phase transfer agents that may be used in embodiments of the present invention include benzalkonium chloride (BAC) and its homologues and analogs of varying carbon chain lengths. Such BAC-like compounds include, but are not limited to, benzalkonium chloride, benzathonium chloride, cetalkonium chloride, cetrimonium bromide, cetylpyridinium chloride, stearalkonium chloride, and the homologues and analogs of these compounds, including various chain lengths of the lipophilic moiety. A BAC homologue with a 4 to 10 carbon lipophilic chain may form ion pairs with 2,2-dimethyl-N,N-dichlorotaurine in aqueous solution with an increased partition into the lipophilic phase. These BAC homologues and analogs are of particular interest as they may possess lower microbiologic activity and may be less irritating to biologic tissues, such as corneal and conjunctival tissues. In general, BAC homologues and analogs with alkyl groups greater than 10 carbons form hydrophobic complexes with N-halogenated amino acids that oil out of aqueous solution and thus may be useful as preservatives for oil-in-water and water-in-oil emulsions such as creams and lotions.

Further phase transfer agents that may be used in embodiments of the present invention include, but are not limited to, phospholipid cholines such as dimyristoylphosphatidylcholine (DMPC).

Phosphonium ion phase transfer agents include but are not limited to tetraalkylphosphonium salts of various alkyl chain lengths from one to 22 carbons, including unsaturated and aromatic alkyl substituents known to those skilled in the art. Salts include but are not limited to chloride, bromide, sulfate, phosphate, borate, and acetate. Examples are tetrabutylphosphonium chloride (TBPC) and benzyldecyldimethylphosphonium chloride.

Preferred combinations of N-halogenated amino acids and phase transfer agents form ion pairs of the following general structure:

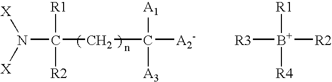

where for the negatively charged portion of the ion pair:

X is chlorine, bromine and/or iodine;

R1 is hydrogen or alkyl, C1-C6;

R2 is hydrogen or alkyl, C1-C6;

R1 and R2 together with the carbon atom to which they attach form a C3-C6 cycloalkyl ring;

n is 0 or an integer from 1-6;

A₁ is hydrogen or alkyl;

A₂ is COO—, SO₃⁻, PO₃⁻, or other acid;

A₃ is hydrogen or alkyl;

and where for the positively charged portion of the ion pair:

B is nitrogen or phosphorous;

R1 to R4 are each selected from alkyl esters, alcohols, hydroxyls, ketones, acids, sulfur-containing and aromatic esters, hydroxyls, ketones, and sulfur-containing acids, and R1 to R4 may not be hydrogen. Further, R1 to R4 should have a carbon atom directly connecting to the nitrogen atom forming a positive charge. This positive charge forms an ion pair with the negatively charged acid moiety of the N-halogenated amino acid.

### III. Applications

Certain formulations described herein may be used to disinfect and/or clean contact lenses in accordance with processes known to those skilled in the art. In a specific application, contact lenses are removed from a patient's eyes and then immersed in contact with formulations described herein for a time sufficient to disinfect the lenses. Disinfection and/or cleaning typically requires soaking the lenses in the formulation for at least 4 to 6 hours.

Embodiments of the invention are usable with many types of contact lenses including, but not limited to, hydrogel soft lenses, HEMA lenses, high water content hydrogel HEMA lenses, and rigid gas permeable (RGP) lenses.

The contacting temperature is preferably in the room temperature range of about 15° C. to about 37° C., but is typically limited by temperatures tolerated by the contact lens material being cleaned or disinfected and/or the stability of the disinfectant or other excipients in the formulation to elevated temperatures.

Although not necessary, the solution containing a contact lens can be agitated, for example, by shaking the container containing the formulation and contact lens to at least facilitate removal of deposit material from the lens. A contact lens optionally may be manually rubbed with saline or a substantially isotonic solution to remove further deposit material from the lens. The cleaning and disinfecting can also include rinsing the lens prior to returning the lens to a wearer's eye.

In situ disinfection and/or cleaning of contact lenses may also be used in certain embodiments of the present invention. In these embodiments, a formulation comprising an N-halogenated amino acid and a phase transfer agent is instilled into the eye of a contact lens wearer. The formulation is applied periodically to ensure acceptable contact lens disinfection and/or cleaning. The wearer optionally may blink or gently rub a closed eyelid to complete the cleaning and disinfection process. The in situ method of the present invention is preferably performed at least daily for soft contact lens applications.

### IV. Formulations

In addition to N-halogenated amino acid and a phase transfer agent, the formulations of the present invention optionally comprise one or more additional components. Such components include, but are not limited to, tonicity agents, preservatives, chelating agents, buffering agents, surfactants, co-solvents, and antioxidants. Other components used in certain embodiments are solubilizing agents, stabilizing agents, comfort-enhancing agents, polymers, emollients, pH-adjusting agents and/or lubricants. Components that may be used in certain formulations of the present invention including water, mixtures of water and water-miscible solvents, such as C1-C7-alkanols, vegetable oils or mineral oils comprising from 0.5 to 5% non-toxic water-soluble polymers, natural products, such as alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinyl methyl ether, polyethylene oxide, preferably cross-linked polyacrylic acid, and mixtures of those products. The concentration of the component is, typically, from 1 to 100,000 times the concentration of the N-halogenated amino acid. In preferred embodiments, components are selected on the basis of their inertness towards the N-halogenated amino acid and/or the phase transfer agent.

In addition to a N-halogenated amino acid, the formulations of the present invention may comprise an additional antimicrobial agent. Suitable antimicrobial agents include, but are not limited to those generally used in contact lens care solutions or in other ophthalmic solutions such as polyquatemium-1, which is a polymeric quaternary ammonium compound, hydrogen peroxide, and potassium iodide.

Suitable antioxidants include, but are not limited to, sulfites, ascorbates, butylated hydroxyanisole (BHA) and butylated hydroxytoluene (BHT).

Surfactants utilized in the formulations of the present invention can be cationic, anionic, nonionic or amphoteric. Preferred surfactants are neutral or noninonic surfactants which may present in amounts up to 5 w/v %. Surfactants that may be used with certain embodiments of the present invention include, but are not limited to, polyethylene glycol ethers or esters of fatty acids, and polyoxypropylene-polyoxyethylene glycol nonionic block copolymers (e.g., poloxamers, such as Pluronic F-127 and F-68).

In certain embodiments of the present invention, suitable cosolvents include glycerin, propylene glycol and polyethylene glycol.

Buffering agents which may be incorporated into formulations of the present invention include, but are not limited to, alkaline metal salts, such as potassium or sodium carbonates, acetates, borates, phosphates, and weak acids, such as acetic acids and boric acids. The preferred buffering agents are alkaline metal borates, such as sodium or potassium borates. Other pH-adjusting agents, such as inorganic acids and bases, may also be utilized. For example, hydrochloric acid or sodium hydroxide may be employed in concentrations suitable for ophthalmic compositions. The above-described buffering agents are generally present in amounts from about 0.1 to about 2.5 w/v %, preferably from about 0.5 to about 1.5% w/v %.

The formulations of the present invention are preferably isotonic, or slightly hypotonic, and generally have an osmolality in the range of 210-320 mOsm/kg, and preferably have an osmolality in the range of 235-300 mOsm/kg. This may require a tonicity agent to bring the osmolality of the formulation to the desired level. Tonicity-adjusting agents include, but are not limited to, sodium chloride, glycerin, sorbitol, or mannitol.

The formulations set forth herein may comprise one or more preservatives. Examples of preservatives include p-hydroxybenzoic acid ester, quaternary ammonium compounds such as, for example, polyquaternium-1, sodium perborate, sodium chlorite, parabens, such as, for example, methylparaben or propylparaben, alcohols, such as, for example, chlorobutanol, benzyl alcohol or phenyl ethanol. In certain embodiments, the formulation may be self-preserved that no preservation agent is required.

In order to effectively disinfect contact lenses and to minimize any side-effects, it is imperative that the disinfection activities of the formulation should be maximized so that a minimum amount of active ingredient is used. It is common knowledge that the activity of these types of antimicrobial agents is the result of the agent itself; the formulation components other than the N-halogenated amino acid normally cause little effect. The amount of the N-halogenated amino acid required to achieve the desired disinfection activity can be determined by persons skilled in the art. The concentration required to achieve the desired activity as a disinfectant while retaining acceptable safety and toxicity properties is referred to herein as “an effective amount”. An effective amount will possess antimicrobial activity sufficient to meet generally accepted standards for activity, such as EN ISO 14729:2001 Ophthalmic optics—Contact lens care products—Microbiological requirements and test methods for products and regimens for hygienic management of contact lenses.

It is also contemplated that the concentrations of the ingredients comprising the formulations of the present invention can vary. In non-limiting aspects, the percentage can be calculated by weight or volume of the total formulation. A person of ordinary skill in the art would understand that the concentrations can vary depending on the addition, substitution, and/or subtraction of ingredients in a given formulation.

The pH of the formulations may be in an ophthalmic acceptable range of 6.7 to 8.0. Accordingly, preferred formulations are prepared using a buffering system that maintains the formulation at a pH of about 6.7 to a pH of about 8.0.

In particular embodiments, formulations are suitable for application to mammalian eyes to disinfect a contact lens in situ. For example, for ophthalmic administration, the formulation may be a solution, a suspension, a gel, water-in-oil and oil-in-water emulsions, or an ointment. Preferred formulations for ophthalmic administration will be aqueous solution in the form of drops. The term “aqueous” typically denotes an aqueous formulation wherein the excipient is >50%, more preferably >75% and in particular >90% by weight water. These drops may be delivered from a single dose ampoule which may preferably be sterile and thus render bacteriostatic components of the formulation unnecessary. Alternatively, the drops may be delivered from a multi-dose bottle which may preferably comprise a device which extracts preservative from the formulation as it is delivered, such devices being known in the art. Additional methods to administer ophthalmic formulations of the present invention may include, but are not limited to, the use of dissolvable inserts comprising an N-halogenated amino acid and a phase transfer agent that are placed beneath the eyelids.

In certain embodiments for in situ disinfection, the N-halogenated amino acid and a phase transfer agent may be formulated in a formulation that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; vinyl polymers, such as polyvinyl alcohol; and carbomers, such as carbomer 934P, carbomer 941, carbomer 940 and carbomer 974P. Ophthalmic formulations for in situ disinfection generally have a viscosity of 0.5-100 cps, preferably 0.5-50 cps, and most preferably 1-20 cps. This relatively low viscosity insures that the product is comfortable, does not cause blurring, and is easily processed during manufacturing, transfer and filling operations.

In certain embodiments of the present invention, a disinfection formulation can be a two-part system. For instance, an N-halogenated amino acid can be present in one container and the remaining formulation components, such as a phase transfer agent, are separated in a separate container or different portion of the same container until a user is ready to use the formulation for disinfection. When needed, the two parts may be mixed by a user and used to disinfect a contact lens. The two-part systems may be useful in cases where one or more components of the formulation have stability problems when combined. One or more components can also have effervescent properties that may, for example, speed up the disintegration and dissolution of a solid portion of the two-part system. Such properties can be conducive to cleaning a contact lens surface, resulting in certain formulations with both cleaning and disinfection activity. Effervescent systems are known to those of skill in the art, and may comprise, for example, sodium bicarbonate plus an acid such as adipic, maleic, or succinic acid.

Formulations of the present invention that comprise cleaning activity in addition to the antimicrobial and/or cleaning activity provided by an N-halogenated amino acid may optionally comprise one or more agents designed to remove protein and other unwanted deposits from contact lens surfaces. Such agents may be oxidizing agents such as sodium chlorite or non-oxidizing agents such as enzymes, detergents, or protein-complex forming agents such chitin or its derivatives.

### V. Examples

The following examples are presented to further illustrate selected embodiments of the present invention.

### Example 1

### Formulation

| Ingredient | % w/v |
|---|---|
| Sodium 2,2-dimethyl-N,N-dichlorotaurine | 0.1 |
| Benzyldecyldimethylammonium Chloride (C10 BAC) | 0.125 |
| Boric Acid | 0.6 |
| Propylene Glycol | 1.0 |
| Pluronic F-68 | 0.05 |
| Sodium Chloride | 0.01 |
| Sodium Hydroxide/Hydrochloric Acid | pH adjust to 7.0* |
| Purified Water | QS |

*The osmolality may be adjusted as necessary to between 210 and 300 mOsm/kg with nonionic osmolality building agents such as propylene glucol or mannitol, or with ionic osmolality building agents such as sodium chloride.

### Example 2

### Formulation

| Ingredient | % w/v |
|---|---|
| Sodium 2,2-dimethyl-N,N-dichlorotaurine | 0.1 |
| Tetrabutylphosphnium chloride (TBPC) | 0.125 |
| Boric Acid | 0.2 |
| Malonic acid* | 1.0 |
| Pluronic P85** | 0.1 |
| Sodium Chloride | 0.01 |
| Sodium Hydroxide/Hydrochloric Acid | pH adjust to 7.0*** |
| Purified Water | QS |

*Other acids like adipic acid may be substituted for malonic acid which do not have alpha-hydroxy groups present. The concentrations of such acids may be adjusted based on their lysozymic removal ability. Such molecules may be used alone or in combination with other acids or in combination with suitable surfactant(s).
**Pluronics are block polymers containing polyethylene oxide and polypropylene oxides. The rations and the type of block may be changed to achieve maximum efficacy. These may be replaced by other compatible surfactants.
***The osmolality may be adjusted as necessary to between 210 and 300 mOsm/kg.

### Example 3

### Antimicrobial Test

Test samples of a formulation comprising a N-halogenated amino acid and a phase transfer agent are prepared at 0.001% target concentrations in vehicles and screened for antimicrobial activity by a time-kill method. The test samples are challenged with standardized suspensions of Candida albicans, Fusarium solani, Pseudomonas aeruginosa, Serratia marcescens and Staphylococcus aureus and the number of surviving microorganisms determined at 6 and 24 hours.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. A method for disinfecting and/or cleaning a contact lens comprising:
contacting a contact lens with a formulation comprising a N-halogenated amino acid and a phase transfer agent for a time sufficient to disinfect and/or clean the lens.

2. The method of claim 1 wherein the phase transfer agent is selected from the group consisting of:
quaternary amines, tetrabutylammonium hydroxide (TBAH), tetrapropylammonium hydroxide (TPAH), hexadecyltrimethylammonium hydroxide, dodecyltriethylammonium hydroxide, tetrabutylphosphonium chloride (TBPC), phosphonium ion phase transfer agents, and combinations thereof.

3. The method of claim 1 wherein said formulation is a two-part formulation.

4. The method of claim 1 wherein the N-halogenated amino acid is a chlorotaurine.

5. The method of claim 4 wherein the chlorotaurine is sodium 2,2-dimethyl-N,N-dichlorotaurine.

6. The method of claim 1 wherein said formulation is free of alpha hydroxyl compounds.

7. A formulation for disinfecting a contact lens comprising:
a N-halogenated amino acid and a phase transfer agent.

8. The formulation of claim 7 wherein the phase transfer agent is selected from the group consisting of:
quaternary amines, tetrabutylammonium hydroxide (TBAH), tetrapropylammonium hydroxide (TPAH), hexadecyltrimethylammonium hydroxide, dodecyltriethylammonium hydroxide, tetrabutylphosphonium chloride (TBPC), phosphonium ion phase transfer agents, and combinations thereof.

9. The formulation of claim 7 wherein said formulation is a two-part formulation.

10. The formulation of claim 7 wherein the N-halogenated amino acid is a chlorotaurine.

11. The formulation of claim 10 wherein the chlorotaurine is sodium 2,2-dimethyl-N,N-dichlorotaurine.

12. The formulation of claim 7 wherein said formulation is free of alpha hydroxyl compounds.

13. A pharmaceutical composition comprising:
a N-halogenated amino acid and a phase transfer agent in a quantity sufficient to preserve said composition.

14. A method for disinfecting or cleaning surfaces comprising:
contacting the surface to be disinfected or cleaned with a formulation comprising a N-halogenated amino acid and a phase transfer agent.

15. The method of claim 14 wherein the surface to be disinfected is a tissue.

16. The method of claim 14 wherein said formulation is free of alpha hydroxyl compounds.
